(19) United States
(12) Patent Application Publication
(10) Pub. No.: US 2019/0388669 A1
(43) Pub. Date: Dec. 26, 2019

(54) MICRONEEDLES MADE FROM POLYCARBONATE-POLYCARBONATE/POLYSILOXANE COPOLYMER COMPOSITIONS

(71) Applicant: SABIC GLOBAL TECHNOLOGIES B.V., Bergen Op Zoom (NL)

(72) Inventors: Maria Dolores MARTINEZ CANOVAS, Murcia (ES); Mark Adrianus Johannes VAN DER MEE, Breda (NL); Robert Dirk VAN DE GRAMPEL, Tholen (NL); Johannes DE BROUWER, Oisterwijk (NL); Herwig JUSTER, Bergen Op Zoom (NL)

(21) Appl. No.: 16/480,801

(22) PCT Filed: Jan. 29, 2018
(86) PCT No.: PCT/IB2018/050537

§ 371 Date: Jul. 25, 2019

(30) Foreign Application Priority Data
Jan. 30, 2017 (EP) 17382041.6

(51) Int. Cl. A61M 37/00; C08L 69/00; C08L 83/04; C08K 5/01; C08L 23/02; C08K 5/103; B29C 39/02; B29C 39/36

(52) U.S. Cl. CPC A61M 37/0015; C08L 69/00; C08L 83/04; C08K 5/01; A61M 2205/0238; C08K 5/103; B29C 39/02; B29C 39/36; C08L 2203/02; C08L 23/02

(57) ABSTRACT

A microneedle comprising a shaft has a proximal end and a distal end and, optionally, a capillary space within said shaft, said capillary space (i) connecting said proximal and distal ends or (ii) extending from the distal end of the shaft and connecting with one or more external openings positioned between the proximal end and distal end or (iii) performing the functions of both (i) and (ii). The microneedle includes a polymer mixture that includes (a) polycarbonate, (b) polycarbonate-polysiloxane copolymer and (c) mold release agent.

### TECHNICAL FIELD

The application concerns microneedles made from polycarbonate-polycarbonate/polysiloxane compositions having high flow, high strength, and good release, and methods of forming same.

### BACKGROUND

Microneedles are attractive for delivery of certain therapeutics. These needles are virtually painless because they do not penetrate deep enough to touch nerves because, unlike traditional syringes and hypodermic needles, microneedles typically only penetrate the outermost layer of the skin. Additionally, shallower penetration reduces the chance of infection and injury. Furthermore, microneedles facilitate delivery of an exact dosage of a therapeutic which allows use lower doses in treatments.

Microneedles often require a manufacturing process that allows mass production at lowest cost, and as a consequence, shortest possible cycle time. In order to have proper transcription of mold texture and shape to the molded part, high flow may be necessary, especially having low viscosity at extremely high shear rates. Furthermore, good release from the production mold is important to reduce cycle time to improve the cost efficiency. Finally, these needles should have good strength to prevent breaking of the microneedle during usage.

Current materials of choice for microneedles are liquid crystalline polymers, polycarbonate, and polyetherimide. These materials all have certain limitations for microneedle applications. Although liquid crystalline polymers have excellent flow, their mechanical properties depend on the flow direction and needle strength may suffer because of this. Polyetherimide is known for its excellent strength, but the flow of this material is far from optimal and very high temperatures are required to be able to mold this polymer into the desired fine features of a microneedle mold. Polycarbonate is flexible in molding conditions, easily formable and has acceptable mechanical properties for the application in microneedles. At high shear rates though, around and beyond 10⁶ inverse seconds (s⁻¹), a plateau value in viscosity may be reached. In some cases, a further increase in shear rate even causes shear thickening behavior which makes filling the fine microfeatures in microneedles molds more difficult. The shear thickening phenomenon is thought to be caused by molecular orientation in the melt.

The fine featured microneedles require excellent mold release properties in order not to get damaged. This can be achieved by cooling the mold deeper than for typical molding operations, but requires an increased expense of energy cost and cycle time and is in general an uneconomic solution. Alternatively, there is a variety of commercial mold release additives such as pentaerythritol tetrastearate (PETS) and waxes that improve mold release behavior, but (traces of) such compounds may remain in the mold and build a deposit after a number of molding cycles which may directly impact needle shape and sharpness.

There is a need in the art for improved compositions that allow for improved performance and economics.

### SUMMARY

Aspects of the disclosure concern microneedles comprising a shaft having a proximal end and a distal end and, optionally, a capillary space within said shaft, said capillary space (i) connecting said proximal and distal ends or (ii) extending from the distal end of the shaft and connecting with one or more external openings positioned between the proximal end and distal end or (iii) performing the functions of both (i) and (ii); said microneedle comprising a polymer mixture which comprises (a) polycarbonate, (b) polycarbonate-polysiloxane copolymer and (c) mold release agent.

Some preferred aspects comprise said capillary space and may be referred to as a porous microneedle. Other preferred aspects, concern microneedles that are solid and do not comprise the capillary space described above. As an example, a solid microneedle may be configured as a transdermal device. Such a transdermal device may be configured to penetrate an epidermis or dermis, or other portion of a patient's body. A surface of the solid microneedle may be coated with a treatment or other coating that may be delivered to the patient as the solid microneedle enters and/or passes through the skin. A solid microneedle, as described herein, may comprise a body having an external surface. Although the body may be porous and may include a passage such as a capillary space, certain aspects of the solid microneedle comprise treatment material disposed on the external surface for delivery to a patient. Treatment material may comprise medical treatment, active pharmaceutical ingredient, minerals, and other materials configured to be dispensed to the patient. Further, the puncture and/or displacement of skin may allow measurements and other operations to be implemented at the puncture or displacement site.

Other aspects concern medical devices suitable for delivery of a therapeutic agent, said device comprising one or more microneedles disclosed herein.

Yet further aspects concern methods of forming a microneedle comprising (a) placing a polymer mixture into a mold, said polymer mixture comprising a polymer mixture which comprises (i) polycarbonate, (ii) polycarbonate-polysiloxane copolymer and (iii) mold release agent; and (b) releasing said polymer mixture from said mold.

### BRIEF DESCRIPTION OF THE FIGURES

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document.

FIG. 1 is a photograph of a microneedle having a bent tip caused during demolding.

### DETAILED DESCRIPTION OF ILLUSTRATIVE ASPECTS

The present disclosure can be understood more readily by reference to the following detailed description of the disclosure and the Examples included therein.

### Microneedle

Microneedles can be used to deliver a therapeutic or to draw bodily fluids (including interstitial fluid) without penetrating tissue as deep a traditional needles. Such microneedles can be used individually or as an array of needles. The size of such needles typically is measured in microns. Some microneedles are between 100 μm (micrometer) and 1 mm (millimeter) in length, preferably between 150 μm and 800 μm.

Certain microneedles are hollow—containing at least one substantially annular bore or channel with a diameter large enough to permit passage of a drug-containing fluid the microneedle. The hollow shafts may be linear—extending from needle base to needle tip. Other microneedles can have a more complex path—for example, extending from the needle base, but then lead to one or more openings on the sides of the needle rather than an opening at the needle tip. Shafts may be tapered or uniform in diameter depending on utility needs.

Other microneedles are solid microneedles—lacking the annular bore or channel described above.

In some aspects, microneedles comprise a shaft having a proximal end and a distal end, a capillary space within said shaft connecting said proximal and distal ends. One utility of microneedles is as part of a medical device that delivers a therapeutic within a patient. Certain medical devices comprise a plurality of microneedles.

Microneedles should have sufficient mechanical strength to remain intact (i) while being inserted into the biological barrier, (ii) while remaining in place for up to a number of days, and (iii) while being removed. Microneedles can be sterilized prior to use.

Microneedles can be manufactured via commercial molding technology. In one aspect, the polymer mixture is supplied in a liquid or flowable state to a mold and allowed to solidify. The solid product is then separated from the mold. Examples of such process are injection molding and hot embossing

### Polycarbonate Polymer

The terms “polycarbonate” or “polycarbonates” as used herein includes copolycarbonates, homopolycarbonates and (co)polyester carbonates.

The term polycarbonate can be further defined as compositions have repeating structural units of the formula (1):

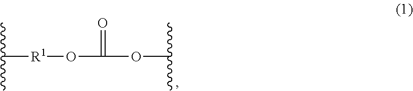

in which at least 60 percent of the total number of IV groups are aromatic organic radicals and the balance thereof are aliphatic, alicyclic, or aromatic radicals. In a further aspect, each IV is an aromatic organic radical and, more preferably, a radical of the formula (2):

—A¹-Y¹Δ²— (2),

wherein each of A¹ and A² is a monocyclic divalent aryl radical and Y¹ is a bridging radical having one or two atoms that separate A¹ from A². In various aspects, one atom separates A¹ from A². For example, radicals of this type include, but are not limited to, radicals such as —O—, —S—, —S(O)—, —S(O₂)—, —C(O)—, methylene, cyclohexyl-methylene, 242.2.11-bicycloheptylidene, ethylidene, isopropylidene, neopentylidene, cyclohexylidene, cyclopentadecylidene, cyclododecylidene, and adamantylidene. The bridging radical Y¹ is preferably a hydrocarbon group or a saturated hydrocarbon group such as methylene, cyclohexylidene, or isopropylidene. Polycarbonate materials include materials disclosed and described in U.S. Pat. Nos. 7,786,246 and 9,096,785, which are hereby incorporated by reference in their entirety for the specific purpose of disclosing various polycarbonate compositions and methods for manufacture of same. Polycarbonate polymers can be manufactured by means known to those skilled in the art.

Apart from the main polymerization reaction in polycarbonate production, there is a series of side reactions consisting of chain rearrangements of the polymer backbone that lead to branching that are often referred to as Fries rearrangement. The Fries species specifically found in bisphenol A melt polycarbonates are the ester type of structures A, B, and C.

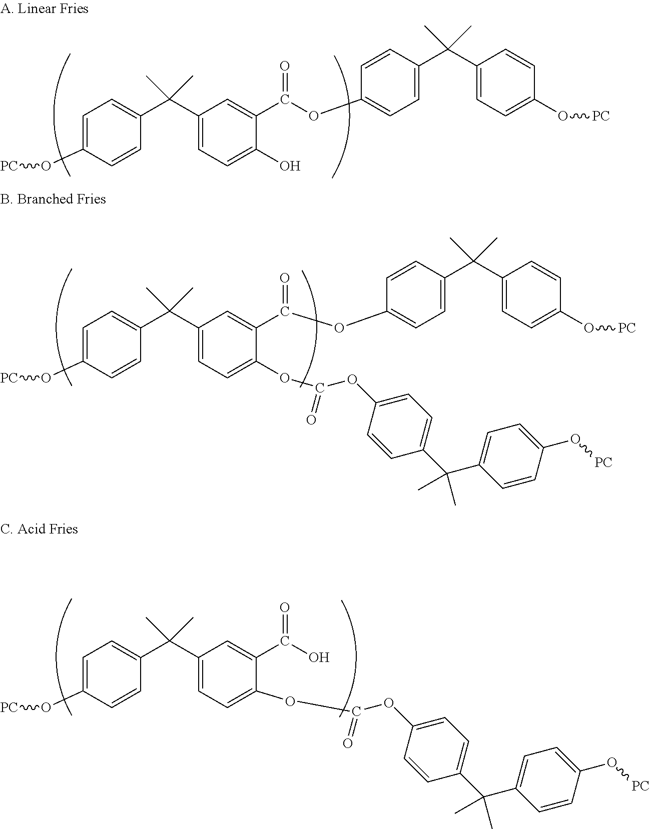

The Fries reaction is induced by the combined effect of basic catalysts, temperature, and residence time, which makes the melt-produced polycarbonates inherently branched as compared with the interfacial polycarbonates since their manufacturing temperatures are lower. Because high branching levels in the resin can have a negative effect on the mechanical properties of the polycarbonate (for example, on impact strength), a product with lower branched Fries product is preferred.

In certain aspects, polycarbonate produced by interfacial polymerization may be utilized. In some processes, bisphenol A and phosgene are reacted in an interfacial polymerization process. Typically, the disodium salt of bisphenol A is dissolved in water and reacted with phosgene which is typically dissolved in a solvent that not miscible with water (such as a chlorinated organic solvent like methylene chloride).

In some aspects, the polycarbonate comprises interfacial polycarbonate having a weight average molecular weight of from about 10,000 Daltons to about 50,000 Daltons, preferably about 15,000 to about 45,000 Daltons. Some interfacial polycarbonates have and endcap level of at least 90% or preferably 95%. Endcap level is the percentage of polymer chains that are capped relative to the total number of chains.

A melt polycarbonate product may also be utilized. The melt polycarbonate process is based on continuous reaction of a dihydroxy compound and a carbonate source in a molten stage. The reaction can occur in a series of reactors where the combined effect of catalyst, temperature, vacuum, and agitation allows for monomer reaction and removal of reaction by-products to displace the reaction equilibrium and effect polymer chain growth. A common polycarbonate made in melt polymerization reactions is derived from bisphenol A (BPA) via reaction with diphenyl carbonate (DPC). This reaction can be catalyzed by, for example, tetra methyl ammonium hydroxide (TMAOH) or tetrabutyl phosphonium acetate (TBPA), which can be added in to a monomer mixture prior to being introduced to a first polymerization unit and sodium hydroxide (NaOH), which can be added to the first reactor or upstream of the first reactor and after a monomer mixer.

The melt polycarbonate may have a molecular weight (Mw) of 20,000 to 120,000 Dalton on a polystyrene basis Polystyrene basis is known to those skilled in the art and refers to liquid chromatography measurements using a polystyrene standard. The melt polycarbonate product may have an endcap level of about 45% to about 80%. Some polycarbonates have an endcap level of about 45% to about 75%, about 55% to about 75%, about 60% to about 70% or about 60% to about 65%. Certain preferred polycarbonates have at least 200 parts per million (ppm) of hydroxide groups. Certain polycarbonates have 200-1100 ppm or 950 to 1050 ppm hydroxide groups.

Polycarbonate polymer may contain endcapping agents. Any suitable endcapping agents can be used provided that such agents do not significantly adversely impact the desired properties of the polycarbonate composition (transparency, for example). Endcapping agents include mono-phenolic compounds, mono-carboxylic acid chlorides, and/or mono-chloroformates. Mono-phenolic endcapping agents are exemplified by monocyclic phenols such as phenol and C₁-C₂₂ alkyl-substituted phenols such as p-cumyl-phenol, resorcinol monobenzoate, and p- and tertiary-butyl phenol; and monoethers of diphenols, such as p-methoxyphenol.

Additionally, some polycarbonates have 900-1100 ppm and 950 to 1050 ppm of Fries products. Fries products include ester type of structures A, B, and C.

### Polycarbonate-Polysiloxane Copolymer

As used herein, the term “polycarbonate-polysiloxane copolymer” is equivalent to polysiloxane-polycarbonate copolymer, polycarbonate-polysiloxane polymer, or polysiloxane-polycarbonate polymer. In various aspects, the polycarbonate-polysiloxane copolymer can be a block copolymer comprising one or more polycarbonate blocks and one or more polysiloxane blocks. In some aspects, the polysiloxane-polycarbonate copolymer comprises polydiorganosiloxane blocks comprising structural units of the general formula (3) below:

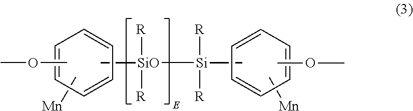

wherein the polydiorganosiloxane block length (E) is from about 20 to about 60; wherein each R group can be the same or different, and is selected from a C₁₋₁₃ monovalent organic group; wherein each M can be the same or different, and is selected from a halogen, cyano, nitro, C₁-C₈ alkylthio, C₁-C₈ alkyl, C₁-C₈ alkoxy, C₂-C₈ alkenyl, C₂-C₈ alkenyloxy group, C₃-C₈ cycloalkyl, C₃-C₈ cycloalkoxy, C₆-C₁₀ aryl, C₆-C₁₀ aryloxy, C₇-C₁₂ aralkyl, C₇-C₁₂aralkoxy, C₇-C₁₂ alkylaryl, or C₇-C₁₂ alkylaryloxy, and where each n is independently 0, 1, 2, 3, or 4. The polysiloxane-polycarbonate copolymer also comprises polycarbonate blocks comprising structural units of the general formula (4) below:

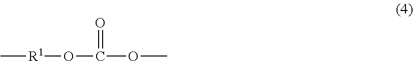

wherein at least 60 percent of the total number of IV groups comprise aromatic moieties and the balance thereof comprise aliphatic, alicyclic, or aromatic moieties.

Certain polysiloxane-polycarbonates materials include materials disclosed and described in U.S. Pat. No. 7,786,246, which is hereby incorporated by reference in its entirety for the specific purpose of disclosing various compositions and methods for manufacture of same.

In some preferred aspects, the polycarbonate-polysiloxane copolymer comprises about 4 to about 30 wt % siloxane. In some copolymers, the amount of siloxane is about 4 to about 15 wt % or about 3.5 to about 22 wt % siloxane or about 4 to about 25 wt %. One preferred aspect comprises about 20 wt % siloxane.

### Release Agent

Examples of mold release agents include both aliphatic and aromatic carboxylic acids and their alkyl esters, for example, stearic acid, behenic acid, pentaerythritol tetrastearate, glycerin tristearate, and ethylene glycol distearate. Polyolefins such as high-density polyethylene, linear low-density polyethylene, low-density polyethylene, and similar polyolefin homopolymers and copolymers can also be used a mold release agents.

Some compositions use pentaerythritol tetrastearate, glycerol monosterate, a wax or a polyalphaolefin.

Mold release agents are typically present in the composition at 0.05 to 10 wt %, based on total weight of the composition, specifically 0.1 to 5 wt %, 0.1 to 1 wt % or 0.1 to 0.5 wt %. Some preferred mold release agents will have high molecular weight, typically greater than 300, to prevent loss of the release agent from the molten polymer mixture during melt processing.

### Additional Components

The additive composition can include an impact modifier, flow modifier, antioxidant, heat stabilizer, light stabilizer, ultraviolet (UV) light stabilizer, UV absorbing additive, plasticizer, lubricant, antistatic agent, anti-fog agent, antimicrobial agent, colorant (e.g., a dye or pigment), surface effect additive, radiation stabilizer, anti-drip agent (e.g., a PTFE-encapsulated styrene-acrylonitrile copolymer (TSAN)), or a combination comprising one or more of the foregoing. For example, a combination of a heat stabilizer, and ultraviolet light stabilizer can be used. In general, the additives are used in the amounts generally known to be effective. For example, the total amount of the additive composition can be 0.001 to 10.0 wt %, or 0.01 to 5 wt %, each based on the total weight of all ingredients in the composition.

The composition can include various additives ordinarily incorporated into polymer compositions of this type, with the proviso that the additive(s) are selected so as to not significantly adversely affect the desired properties of the thermoplastic composition (good compatibility for example). Such additives can be mixed at a suitable time during the mixing of the components for forming the composition.

Examples of impact modifiers include natural rubber, fluoroelastomers, ethylene-propylene rubber (EPR), ethylene-butene rubber, ethylene-propylene-diene monomer rubber (EPDM), acrylate rubbers, hydrogenated nitrile rubber (HNBR), silicone elastomers, styrene-butadiene-styrene (SBS), styrene-butadiene rubber (SBR), styrene-(ethylene-butene)-styrene (SEBS), acrylonitrile-butadiene-styrene (ABS), acrylonitrile-ethylene-propylene-diene-styrene (AES), styrene-isoprene-styrene (SIS), styrene-(ethylene-propylene)-styrene (SEPS), methyl methacrylate-butadiene-styrene (MBS), high rubber graft (HRG), and the like. Some suitable impact modifies include PC(polycarbonate)/ABS (such as Cycoloy PC/ABS) and MBS type formulations.

Heat stabilizer additives include organophosphites (e.g. triphenyl phosphite, tris-(2,6-dimethylphenyl)phosphite, tris-(mixed mono- and di-nonylphenyl)phosphite or the like), phosphonates (e.g., dimethylbenzene phosphonate or the like), phosphates (e.g., trimethyl phosphate, or the like), or combinations comprising at least one of the foregoing heat stabilizers. The heat stabilizer can be tris(2,4-di-t-butylphenyl) phosphate available as IRGAPHOS™ 168. Heat stabilizers are generally used in amounts of 0.01 to 5 wt %, based on the total weight of polymer in the composition.

There is considerable overlap among plasticizers, lubricants, and mold release agents, which include, for example, glycerol tristearate (GTS), phthalic acid esters (e.g., octyl-4,5-epoxy-hexahydrophthalate), tris-(octoxycarbonylethyl)isocyanurate, tristearin, di- or polyfunctional aromatic phosphates (e.g., resorcinol tetraphenyl diphosphate (RDP), the bis(diphenyl) phosphate of hydroquinone and the bis(diphenyl) phosphate of bisphenol A); poly-alpha-olefins; epoxidized soybean oil; silicones, including silicone oils (e.g., poly(dimethyl diphenyl siloxanes); esters, for example, fatty acid esters (e.g., alkyl stearyl esters, such as, methyl stearate, stearyl stearate, and the like), waxes (e.g., beeswax, montan wax, paraffin wax, or the like), or combinations comprising at least one of the foregoing plasticizers, lubricants, and mold release agents. These are generally used in amounts of 0.01 to 5 wt %, based on the total weight of the polymer in the composition.

Light stabilizers, in particular ultraviolet light (UV) absorbing additives, also referred to as UV stabilizers, include hydroxybenzophenones (e.g., 2-hydroxy-4-n-octoxy benzophenone), hydroxybenzotriazines, cyanoacrylates, oxanilides, benzoxazinones (e.g., 2,2′-(1,4-phenylene)bis(4H-3,1-benzoxazin-4-one, commercially available under the trade name CYASORB UV-3638 from Cytec), aryl salicylates, hydroxybenzotriazoles (e.g., 2-(2-hydroxy-5-methylphenyl)benzotriazole, 2-(2-hydroxy-5-tert-octylphenyl)benzotriazole, and 2-(2H-benzotriazol-2-yl)-4-(1,1,3,3-tetramethylbutyl)-phenol, commercially available under the trade name CYASORB 5411 from Cytec) or combinations comprising at least one of the foregoing light stabilizers. The UV stabilizers can be present in an amount of 0.01 to 1 wt %, specifically, 0.1 to 0.5 wt %, and more specifically, 0.15 to 0.4 wt %, based upon the total weight of polymer in the composition.

Antioxidant additives include organophosphites such as tris(nonyl phenyl)phosphite, tris(2,4-di-t-butylphenyl)phosphite, bis(2,4-di-t-butylphenyl)pentaerythritol diphosphite, distearyl pentaerythritol diphosphite; alkylated monophenols or polyphenols; alkylated reaction products of polyphenols with dienes, such as tetrakis[methylene(3,5-di-tert-butyl-4-hydroxyhydrocinnamate)] methane; butylated reaction products of para-cresol or dicyclopentadiene; alkylated hydroquinones; hydroxylated thiodiphenyl ethers; alkylidene-bisphenols; benzyl compounds; esters of beta-(3,5-di-tert-butyl-4-hydroxyphenyl)-propionic acid with monohydric or polyhydric alcohols; esters of beta-(5-tert-butyl-4-hydroxy-3-methylphenyl)-propionic acid with monohydric or polyhydric alcohols; esters of thioalkyl or thioaryl compounds such as distearylthiopropionate, dilaurylthiopropionate, ditridecylthiodipropionate, octadecyl-3-(3,5-di-tert-butyl-4-hydroxyphenyl)propionate, pentaerythrityl-tetrakis[3-(3,5-di-tert-butyl-4-hydroxyphenyl)propionate; amides of beta-(3,5-di-tert-butyl-4-hydroxyphenyl)-propionic acid, or combinations comprising at least one of the foregoing antioxidants. Antioxidants are used in amounts of 0.01 to 0.1 parts by weight, based on 100 parts by weight of the total composition, excluding any filler.

Anti-drip agents can also be used in the composition, for example a fibril forming or non-fibril forming fluoropolymer such as polytetrafluoroethylene (PTFE). The anti-drip agent can be encapsulated by a rigid copolymer, for example styrene-acrylonitrile copolymer (SAN). PTFE encapsulated in SAN is known as TSAN. A TSAN comprises 50 wt % PTFE and 50 wt % SAN, based on the total weight of the encapsulated fluoropolymer. The SAN can comprise, for example, 75 wt % styrene and 25 wt % acrylonitrile based on the total weight of the copolymer. Antidrip agents can be used in amounts of 0.1 to 10 parts by weight, based on 100 parts by weight of the total composition, excluding any filler.

### Polymer Mixtures

Some polymer mixtures used in the disclosure comprise:
about 30 to about 89.5 wt % polycarbonate (PC);
about 10 to about 69.5 wt % polycarbonate-polysiloxane copolymer; and
about 0.1 to about 0.5 wt % mold release agent;
wherein all wt % values are based on the total weight of the polymer mixture.

Other mixtures comprise about 70 to about 89.5 wt % polycarbonate and about 10 to about 30 wt % polycarbonate-polysiloxane copolymer.

The polymer mixture preferably has a melt volume flow rate (MVR) of greater than 30 cubic centimeters per minute (cm³/min), 35 cm³/min, 40 cm³/min, as measured according to ASTM 1133 at 300 degrees Celsius (° C.) and 1.2 kilogram (kg).

The polymer mixture preferably has an Izod Notched Impact of at least 35 kilojoule per square meter (kJ/m²) or more preferably at least 40 kJ/m² as measured according to ISO 180-1A.

The polymer mixture preferably has a heat deflection temperature (HDT) of at least 115° C. on a 3.2 millimeter (mm) sample at 1.8 megapascals (MPa) as measured according to ASTM D648.

In addition, the polymer mixture preferably exhibits excellent release, as measured by ejection force (N) and coefficient of friction. Release performance is significantly better for the compositions of the disclosure compared with than for commercial alternatives, including high flow PC, impact modified PC and standard flow Lexan™ EXL.

The polymer mixtures also preferably show (i) high flow at high shear conditions to allow good transcription of mold texture and excellent filling of the finest mold features, (ii) good strength and impact (as indicated by ductile Izod Notched Impact at room temperature and modulus), and (iii) high release to have efficient de-molding and reduced cooling and cycle time during molding.

### Definitions

It is to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

“ppm” refers to parts per million.

As used herein the terms “weight percent,” “weight %,” and “wt %” of a component, which can be used interchangeably, unless specifically stated to the contrary, are based on the total weight of the formulation or composition in which the component is included. For example if a particular element or component in a composition or article is said to have 8% by weight, it is understood that this percentage is relative to a total compositional percentage of 100% by weight.

As used herein, the terms “weight average molecular weight” or “Mw” can be used interchangeably, and are defined by the formula:

$${M}_{w} = \frac{\sum {N}_{i} {M}_{i}^{2}}{\sum {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards. Polystyrene basis refers to measurements using a polystyrene standard.

The term “siloxane” refers to a segment having a Si—O—Si linkage.

The term “flowable” means capable of flowing or being flowed. Typically a polymer is heated such that it is in a melted state to become flowable.

° C. is degrees Celsius. μm is micrometer. cS is centistroke. kG is kilogram.

“Interfacial polycarbonate” is produced by a process where typically the disodium salt of bisphenol A (BPA) is dissolved in water and reacted with phosgene which is typically dissolved in a solvent that not miscible with water.

“Melt polycarbonate” is produced by a process where BPA reacts with diphenyl carbonate (DPC) in a molten state without the solvent.

Izod Notched Impact tests are performed according to ISO 180-1A.

Melt Volume Flow Rate (MVR) is measured according to ASTM 1133 at 300° C. and 1.2 kg.

Heat deflection temperature (HDT) is measured using a 3.2 mm sample at 1.8 MPa as measured according to ISO 75A.

Ejection force was measured by injection molding sleeves in a core and then measuring the force necessary to remove the sleeve from the core. At the opening of the mold, the sleeve remained on the core due to the material contraction. At the end of the opening stroke, the ejector pins detached the sleeve from the core. The force applied to the sleeve for demolding was measured as the ejection force. The surface temperature of the sleeve was kept constant so that an accurate comparison of ejection force could be made. Ejection force was measured with a composition formed into the exemplary sleeve/mold. It should be noted that other sleeves/molds with other dimensions could be used for comparing the compositions of the disclosure to the reference compositions. Measurement process parameters were as follows.

| | |
|---|---|
| Melt temperature | 300° C. |
| Surface temperature of the | 95° C. |
| core | |
| Ejection speed | 70 millimeter per |
| | second (mm/s) |
| Cycle time | 20.1 second (s) |
| Overall cooling time | 13.6 s |
| Pre-drying | 120° C., 4 hours (h) |
| Max. injection pressure | 1000 bar |
| Injection speed | 25 cm³/s |
| Changeover pressure | 100 bar |
| Temperature injection plate | 89° C. |
| IM3 | 97° C. |

Coefficient of friction was measured according to UL 410. A specialized mold insert was designed for the measurement of the CoF. A concave disc was molded and after a fixed cooling time, the mold opened slightly to allow rotation of the disc while maintaining a constant normal pressure on the part. The friction core exerted a constant pressure onto the disc. The disc itself was rotated by an electro-motor driven belt, while the resulting torque of the disc onto the friction core was measured. By the concave shape of the disc, the contact surface area is limited to an outer ring with a fixed surface area. For each material, the coefficient of friction is determined as the average over 10 discs/measurements. It should be noted that other discs/molds with other dimensions could be used for comparing the compositions of the disclosure to the reference compositions. Measurement process parameters were as follows.

| | |
|---|---|
| Molding machine | Arburg 370 |
| Screw diameter | 25 mm |
| Injection speed | 40 mm/s |
| Drying | Vacuum drying (120° C., 5 hrs) |
| | or hot air drying (120° C., 3 hrs) |
| Melt temperature | 300° C. |
| Mold temperature | 90° C. |
| Reference material | BMS Makrolon 2808 |

### Aspects

The present disclosure comprises at least the following aspects.

Aspect 1. A microneedle comprising a shaft having a proximal end and a distal end and, optionally, a capillary space within said shaft, said capillary space (i) connecting said proximal and distal ends or (ii) extending from the distal end of the shaft and connecting with one or more external openings positioned between the proximal end and distal end or (iii) performing the functions of both (i) and (ii), said microneedle comprising a polymer mixture which comprises (a) polycarbonate, (b) polycarbonate-polysiloxane copolymer and (c) mold release agent.

Aspect 2. The microneedle of Aspect 1, configured a solid transdermal microneedle having a treatment material disposed on an exterior surface of a body of the solid transdermal microneedle.

Aspect 3. The microneedle of Aspect 1 or Aspect 2 comprising:
about 30 to about 89.5 wt % polycarbonate;
about 10 to about 69.5 wt % polycarbonate-polysiloxane copolymer; and
about 0.1 to about 0.5 wt % mold release agent;
wherein all wt % values are based on the total weight of the polymer mixture.

Aspect 4. The microneedle of any one of Aspects 1-3, wherein said polymer mixture has a melt volume flow rate (MVR) of greater than 35 cm³/min as measured according to ASTM 1133 at 300° C. and 1.2 kg.

Aspect 5. The microneedle of any one of Aspects 1-3, wherein said polymer mixture has a melt volume flow rate (MVR) of greater than 40 cm³/min as measured according to ASTM 1133 at 300° C. and 1.2 kg.

Aspect 6. The microneedle of any one of Aspects 1-5, wherein said polymer mixture has a Izod Notched Impact of at least 35 kJ/m² as measured according to ISO 180-1A.

Aspect 7. The microneedle of any of Aspects 1-6, wherein the polycarbonate-polysiloxane copolymer comprises about 4 to about 40 wt % siloxane.

Aspect 8. The microneedle of any of Aspects 1-6, wherein the polycarbonate-polysiloxane copolymer comprises about 10 to about 30 wt % siloxane.

Aspect 9. The microneedle of any one of Aspects 1-8, wherein said polymer mixture comprises about 70 to about 89.5 wt % polycarbonate and about 10 to about 30 wt % polycarbonate-polysiloxane copolymer.

Aspect 10. The microneedle of any one of Aspects 1-9, wherein said polycarbonate comprises interfacial polycarbonate.

Aspect 11. The microneedle of any one of Aspects 1-9, wherein said polycarbonate comprises melt polycarbonate.

Aspect 12. The microneedle of any one of Aspects 1-11, wherein said polycarbonate has a weight average molecular weight of from about 10,000 Daltons to about 35,000 Daltons.

Aspect 13. The microneedle of any one of Aspects 1-12, wherein said mold release agent comprises one or more of pentaerythritol stearate, glycerol monostearate, a wax or a polyalphaolefin.

Aspect 14. The microneedle of any one of Aspects 1-13, wherein said microneedle consists of said polymer mixture.

Aspect 15. The microneedle of any one of Aspects 1-14, wherein said polymer mixture has a heat deflection temperature (HDT) of at least 115° C. on a 3.2 mm sample at 1.8 MPa as measured according to ISO 75A.

Aspect 16. The microneedle of any one of Aspects 1 to 15, wherein the polymer mixture has a coefficient of friction of less than about 20 as determined by UL410.

Aspect 17. The microneedle of anyone of anyone of Aspects 1 to 16, wherein a demolding ejection force of less than about 400 N, or less than about 350 N, or less than about 300 N, or less than about 250 N is required to remove the molded polymer mixture from the mold.

Aspect 18. A medical device suitable for delivery of a therapeutic agent, said device comprising one or more microneedles of Aspects 1-17.

Aspect 19. A method of forming a microneedle comprising:

(a) placing a polymer mixture into a mold, said polymer mixture comprising (i) polycarbonate, (ii) polycarbonate-polysiloxane copolymer and (iii) mold release agent, said polymer mixture being a temperature such that it is flowable;

(b) allowing said polymer to solidify; and

(c) releasing said solidified polymer mixture from said mold.

Aspect 20. The method of Aspect 19, wherein said polymer mixture comprising:
30 to about 89.5 wt % polycarbonate;
about 10 to about 69.5 wt % polycarbonate-polysiloxane copolymer; and
about 0.1 to about 0.5 wt % mold release agent;
wherein all wt % values are based on the total weight of the polymer mixture.

Aspect 21. The method of Aspect 19 or Aspect 20, wherein said polymer mixture has a melt volume flow rate (MVR) of greater than 35 cm³/min as measured according to ASTM 1133 at 300° C. and 1.2 kg.

Aspect 22. The method of Aspect 19 or Aspect 20, wherein said polymer mixture has a melt volume flow rate (MVR) of greater than 40 cm³/min as measured according to ASTM 1133 at 300° C. and 1.2 kg.

Aspect 23. The method of Aspect 19 or Aspect 20, wherein said polymer mixture has a melt volume flow rate (MVR) of greater than 50 cm³/min as measured according to ASTM 1133 at 300° C. and 1.2 kg.

Aspect 24. The method of any one of Aspects 18-23, wherein the polycarbonate-polysiloxane copolymer comprises about 4 to about 30 wt % siloxane.

Aspect 25. The method of any one of Aspects 18-24, wherein said polycarbonate comprises interfacial polycarbonate.

Aspect 26. The method of any one of Aspects 18-25, wherein said polycarbonate comprises melt polycarbonate.

Aspect 27. The method of any one of Aspects 18-26, wherein aid mold release agent comprises one or more of pentaerythritol stearate, glycerol monostearate, a wax or a polyalphaolefin.

Aspect 28. The method of any one of Aspects 18-27, wherein said polymer mixture comprises about 70 to about 89.5 wt % polycarbonate and about 10 to about 30 wt % polycarbonate-polysiloxane copolymer.

Aspect 29. The method of any one of Aspects 18-28, further comprising removing the article from the mold, wherein the removal requires an ejection force at least about 50% lower than the ejection force required for a substantially identical reference article without a polycarbonate-polysiloxane copolymer.

Aspect 30. The method of any one of Aspects 18-28, wherein the coefficient of friction for the removal is at least about 15% lower than the coefficient of friction for removal of a substantially identical reference article without a polycarbonate-polysiloxane copolymer.

### EXAMPLES

The disclosure is illustrated by the following non-limiting examples.

A number of polymer blends were made and summarized in Table 2.

The polymer blends of Table 2 can be molded into microneedles using conventional technology. Such microneedles can be integrated into medical devices used for drawing blood from a patient or delivery of therapeutic agents to a patient.

PC-PS1 is a transparent BPA polycarbonate-polydimethylsiloxane block copolymer comprising about 6 wt % siloxane (PDMS residues). PC-PS1 has an Mw of 22,500-23,500 g/mol in PC equivalent units (measured on a size exclusion column calibrated with broad molar mass polycarbonate standards of known mass determined through light scattering). PC-PS1 is made through an interfacial polymerization process using para-cumyl phenol as end-cap.

PC-PS2 is an opaque BPA polycarbonate-polydimethylsiloxane block copolymer comprising about 20 wt % of siloxane (PDMS residues). PC-PS2 has an Mw of 30,000-31,000 g/mol in PC equivalent units (measured on a size exclusion column calibrated with broad molar mass polycarbonate standards of known mass determined through light scattering). PC-PS2 is made through an interfacial polymerization process using para-cumyl phenol as end-cap.

PC1 is an optical quality BPA polycarbonate. PC1 has an Mw of 18,400-19,000 g/mol in PC equivalent units (measured on a size exclusion column calibrated with broad molar mass polycarbonate standards of known mass determined through light scattering). PC1 is made through an interfacial polymerization process using para-cumyl phenol as end-cap. MVR measured at 300° C. and 1.2 kg is 60 to 85.

PC2 is a BPA polycarbonate. PC2 is made using a melt bulk polymerization process. Branched Bisphenol A polycarbonate homopolymer. Mw of about 18,000 g/mol as determined by GPC using polycarbonate standards for calibration. Fries level 250-350 ppm. BPA/Phenol end-capped.

PC3 is a BPA polycarbonate. PC3 is made using a melt bulk polymerization process. Branched Bisphenol A polycarbonate homopolymer. Mw of about 20,600 g/mol as determined by GPC using polycarbonate standards for calibration. Fries level 350 ppm. BPA/Phenol end-capped.

Tospearl™ 120 is a microfine silicone resin.

TABLE 1
Description of Example Materials.

| Code | Description | Type | CAS | |
|---|---|---|---|---|
| PC-PS1 | transparent PC-siloxane | polymer | 202483-49-6 | |
| | copolymer (6% w siloxane) | | | |
| PC-PS2 | opaque PC-siloxane | polymer | 202483-49-6 | |
| | copolymer (6% w siloxane) | | | |
| PC1 | high flow polycarbonate | polymer | 111211-39-3 | |
| | (interfacial process) | | | |
| PC2 | high flow polycarbonate | polymer | 25929-04-8 | |
| | (melt process) | | | |
| TDBPP | Tris(di-t- | heat | 31570-04-4 | tris(2,4-di |
| | butylphenyl)phosphite | stabilizer | | tert•butylphenyl) |
| | | | | phosphite |
| PAO | polyalphaolefin wax | release aid | 68037-01-4 | 1-decene, tri-, |
| | | | | tetra-,pentamer, |
| | | | | hydrogenated |
| PETS | PETS | release aid | 115-83-3 | pentaerythritol |
| | | | | tetrastearate |

### Examples 1-18

Examples 1-21 were produced accruing to Table 2. All values are in weight percent (wt %) based on the total weight of the particular example.

### Examples 19-38

Examples 19-36 are prepared by molding the polymer blends of Examples 1-21 into microneedles.

TABLE 2
Compositions of Examples 1-21

| | | Ex 1 | Ex 2 | Ex 3 | Ex 4 | Ex 5 | Ex 6 | Ex 7 | Ex 8 | Ex 9 | Ex 10 | Ex 11 |
|---|---|---|---|---|---|---|---|---|---|---|---|---|
| PC-PS1 | % w | 37.5 | | 37.5 | | 50 | | 66.7 | | | | |
| PC-PS2 | % w | | 11.25 | | 11.25 | | 15 | | 20 | 11.25 | 20 | 11.25 |
| PC1 | % w | 47.09 | 73.34 | | | 34.59 | 69.59 | 17.89 | 64.59 | 73.29 | 64.54 | 68.19 |
| PC2 | % w | | | 47.09 | 73.34 | | | | | | | |
| PC3 | % w | | | | | | | | | | | |
| TDBPP | % w | 0.06 | 0.06 | 0.06 | 0.06 | 0.06 | 0.06 | 0.06 | 0.06 | 0.06 | 0.06 | 0.06 |
| PAO | % w | 0.35 | 0.35 | 0.35 | 0.35 | 0.35 | 0.35 | 0.35 | 0.35 | 0.35 | 0.35 | 0.35 |
| PETS | % w | | | | | | | | | | | |
| EPOXY | % w | | | | | | | | | 0.05 | 0.05 | |
| TALC | % w | | | | | | | | | | | 5 |
| H3PO3 | % w | | | | | | | | | | | 0.15 |
| FUMED SILICA | % w | | | | | | | | | | | |
| TOSPEARL 120 | % w | | | | | | | | | | | |

| | | Ex 12 | Ex 13 | Ex 14 | Ex 15 | Ex 16 | Ex 17 | Ex 18 | Ex 19 | Ex 20 | Ex 21 |
|---|---|---|---|---|---|---|---|---|---|---|---|
| PC-PS1 | % w | | | | | | | | | | |
| PC-PS2 | % w | 20 | 11.25 | 20 | 11.25 | 20 | 11.25 | 20 | 11.25 | 20 | |
| PC1 | % w | 69.44 | 80.34 | 61.59 | 71.34 | 62.59 | 73.34 | 64.59 | 73.28 | 64.53 | |
| PC2 | % w | | | | | | | | | | 5 |
| PC3 | % w | | | | | | | | | | 94.94 |
| TDBPP | % w | 0.06 | 0.06 | 0.06 | 0.06 | 0.06 | 0.06 | 0.06 | 0.12 | 0.12 | 0.06 |
| PAO | % w | 0.35 | 0.35 | 0.35 | 0.35 | 0.35 | | | 0.35 | 0.35 | |
| PETS | % w | | | | | | 0.35 | 0.35 | | | |
| EPOXY | % w | | | | | | | | | | |
| TALC | % w | 5 | | | | | | | | | |
| H3PO3 | % w | 0.15 | | | | | | | | | |
| FUMED SILICA | % w | | 3 | 3 | | | | | | | |
| TOSPEARL 120 | % w | | | | 2 | 2 | | | | | |

TABLE 3
Results

| | | Ex 1 | Ex 2 | Ex 3 | Ex 4 | Ex 5 | Ex 6 | Ex 7 | Ex 8 | Ex 9 | Ex 10 | Ex 11 |
|---|---|---|---|---|---|---|---|---|---|---|---|---|
| Part of Disclosure | | | Y | | Y | | | | | | | |
| Silicone content | % w | 2.25 | 2.25 | 2.25 | 2.25 | 3.00 | 3.00 | 4.00 | 4.00 | 2.25 | 4.00 | 2.25 |
| MVR | cm³/10 min | 32 | 39 | 36 | 44 | 26 | 35 | 19 | 30 | 40 | 33 | 38 |
| Izod Notched Impact | kJ/m2 | 41 | 47 | 34 | 39 | 45 | 47 | 50 | 48 | 45 | 48 | 8 |
| HDT | ° C. | 118 | 121 | 118 | 119 | 118 | 121 | 116 | 120 | 120 | 121 | 121 |
| Viscosity @ | Pa · s | 53.4 | 48.8 | 51.8 | 46.4 | 55.9 | 50.2 | 60.0 | 50.6 | 49.2 | 51.2 | 46.8 |
| 300° C., 10000 s−1 | | | | | | | | | | | | |
| Mw | kDa | 20.4 | 19.9 | 20.3 | 19.6 | 20.9 | 20.3 | 21.7 | 20.7 | 19.9 | 20.7 | 19.8 |
| Ejection Force | N | 398 | 269 | 293 | 237 | 331 | 247 | 322 | 215 | 218 | | |
| Friction Coefficient | [—] | 17 | 13 | 14 | 11 | 18 | 13 | 18 | 18 | 19 | | |

| | | Ex 12 | Ex 13 | Ex 14 | Ex 15 | Ex 16 | Ex 17 | Ex 18 | Ex 19 | Ex 20 | Ex 21 |
|---|---|---|---|---|---|---|---|---|---|---|---|
| Part of Disclosure | | | Y | | Y | | | | Y | | |
| Silicone content | % w | 4.00 | 2.25 | 4.00 | 2.25 | 4.00 | 2.25 | 4.00 | 2.25 | 4.00 | 0.00 |
| MVR | cm³/10 min | 30 | 39 | 28 | 38 | 28 | 40 | 30 | 42 | 30 | 30 |
| Izod Notched Impact | kJ/m2 | 11 | 38 | 40 | 42 | 44 | 44 | 49 | 45 | 48 | 35 |
| HDT | ° C. | 122 | 120 | 120 | 120 | 121 | 121 | 120 | 120 | 120 | 120 |
| Viscosity @ | Pa · s | 48.2 | 49.5 | 52.2 | 49.9 | 51.8 | 48.6 | 51.8 | 49.2 | 51.2 | |
| 300° C., 10000 s−1 | | | | | | | | | | | |
| Mw | kDa | 20.5 | 19.7 | 20.5 | 20.0 | 20.8 | 19.9 | 20.6 | 19.9 | 20.7 | |
| Ejection Force | N | | 239 | | 267 | | 448 | | 297 | | 646 |
| Friction Coefficient | [—] | | 12 | | 13 | | 15 | | 15 | | 49 |

The results show that pure high flow PC (Ex 21) without any release agent has a high ejection force and high friction coefficient.

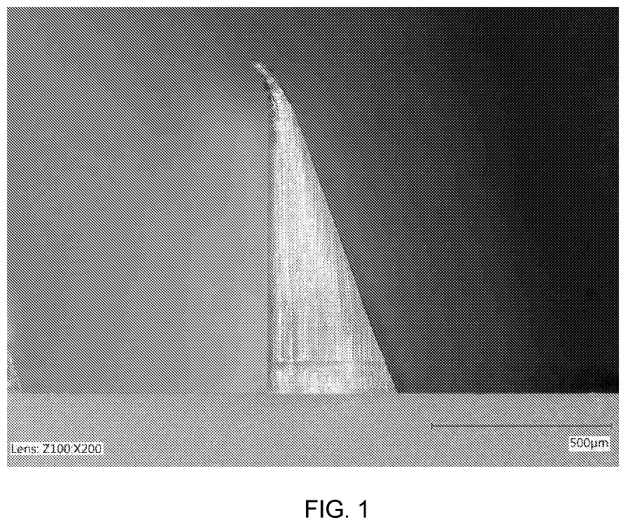

The ejection force and friction coefficient go down in a commercial blend of PC and PC-PS copolymer that also contains PAO release agent (Ex 1). It was found however that during demolding, these forces may still damage the microneedle resulting in bended tips and reduced efficiency in the application (FIG. 1).

Friction and injection force can be further improved when a different polycarbonate-polysiloxane copolymer is used. PC-PS2 contains 20% w siloxane which results in the formation of larger siloxane domains when blended with PC than when PC-PS1 is used which contains 6% w siloxane. (Ex. 2 and Ex. 4).

This PC-PS2 is effective when blended both with interfacially produced (as in Ex. 2) or melt produced (Ex. 4) polycarbonate homopolymer. The latter has a further advantage of lower viscosity and higher MVR, making processing easier.

The results further show than increasing the siloxane level through a higher level of PC-PS2 reduces the ejection force, but not further improves the friction coefficient (series Ex. 1 Ex. 5 Ex 7 and series Ex. 2 Ex. 6 Ex 8). The formulations are tolerant towards most other additives that have been studied (Ex. 13 to Ex 20).

Surprisingly the use of polyalphaolefin as a release agent has a better result than the use of pentaerythritol tetrastearate as evidenced by the much higher ejection force required to eject Ex. 17 compared to Ex. 2.

1. A microneedle comprising a shaft having a proximal end and a distal end and, optionally, a capillary space within said shaft,
said capillary space (i) connecting said proximal end and said distal end or (ii) extending from the distal end of the shaft and connecting with one or more external openings positioned between the proximal end and distal end or (iii) performing the functions of both (i) and (ii),
said microneedle comprising a polymer mixture which comprises (a) polycarbonate, (b) polycarbonate-polysiloxane copolymer and (c) mold release agent.

2. The microneedle of claim 1, wherein said microneedle is configured as a solid transdermal microneedle having a treatment material disposed on an exterior surface of a body of the solid transdermal microneedle.

3. The microneedle of claim 1 comprising:
about 30 to about 89.5 wt % polycarbonate;
about 10 to about 69.5 wt % polycarbonate-polysiloxane copolymer; and
about 0.1 to about 0.5 wt % mold release agent,
wherein all wt % values are based on the total weight of the polymer mixture.

4. The microneedle of claim 1, wherein said polymer mixture has a melt volume flow rate (MVR) of greater than 35 cubic centimeters per minute (cm³/min) as measured according to ASTM 1133 at 300 degrees Celsius (° C.) and 1.2 kilogram (kg).

5. The microneedle of claim 1, wherein said polymer mixture has an Izod Notched Impact of at least 35 kilojoules per square meter (kJ/m²) as measured according to ISO 180-1A.

6. The microneedle of claim 1, wherein the polycarbonate-polysiloxane copolymer comprises about 4 to about 40 wt % siloxane based on the weight of the copolymer.

7. The microneedle of claim 1, wherein said polymer mixture comprises about 70 to about 89.5 wt % polycarbonate and about 10 to about 30 wt % polycarbonate-polysiloxane copolymer.

8. The microneedle of claim 1, wherein said polycarbonate comprises interfacial polycarbonate.

9. The microneedle of claim 1, wherein said polycarbonate comprises melt polycarbonate.

10. The microneedle of claim 1, wherein said mold release agent comprises one or more of pentaerythritol stearate, glycerol monostearate, a wax or a polyalphaolefin.

11. The microneedle of claim 1, wherein said microneedle consists of said polymer mixture.

12. The microneedle of claim 1, wherein said polymer mixture has a heat deflection temperature (HDT) of at least 115° C. on a 3.2 millimeter (mm) sample at 1.8 megapascals (MPa) as measured according to ISO 75A.

13. A medical device suitable for delivery of a therapeutic agent, said medical device comprising one or more microneedles of claim 1.

14. A method of forming a microneedle comprising:
(a) placing a polymer mixture into a mold, said polymer mixture comprising (i) polycarbonate, (ii) polycarbonate-polysiloxane copolymer and (iii) mold release agent, said polymer mixture being a temperature such that it is flowable;
(b) allowing said polymer to solidify and form a solidified polymer mixture; and
(c) releasing said solidified polymer mixture from said mold.

15. The method of claim 14, wherein said polymer mixture comprises:
about 30 to about 89.5 wt % polycarbonate;
about 10 to about 69.5 wt % polycarbonate-polysiloxane copolymer; and
about 0.1 to about 0.5 wt % mold release agent,
wherein all wt % values are based on the total weight of the polymer mixture.

16. The method of claim 14, wherein said polymer mixture has a melt volume flow rate (MVR) of greater than 35 cm³/min as measured according to ASTM 1133 at 300° C. and 1.2 kg.

17. The method of claim 14, wherein the polycarbonate-polysiloxane copolymer comprises about 4 to about 40 wt % siloxane.

18. The method of claim 14, wherein said polycarbonate comprises interfacial polycarbonate.

19. The method of claim 14, wherein said polycarbonate comprises melt polycarbonate.

20. The method of claim 14, wherein said mold release agent comprises one or more of pentaerythritol stearate, glycerol monostearate, a wax or a polyalphaolefin.
